(19) United States
(12) Patent Application Publication
(10) Pub. No.: US 2024/0424489 A1
(43) Pub. Date: Dec. 26, 2024

(54) PINCER-BASED COBALT CATALYSTS FOR HYDROGENATION REACTION AND ITS PROCESS OF PREPARATION THEREOF

(71) Applicant: Council of Scientific & Industrial Research, New Delhi (IN)

(72) Inventors: Benudhar Punji, Pune (IN); Dipesh Mamraj Sharma, Pune (IN)

(21) Appl. No.: 18/709,574

(22) PCT Filed: Nov. 4, 2022
(86) PCT No.: PCT/IN2022/050971

§ 371 Date: May 13, 2024

(30) Foreign Application Priority Data
Nov. 15, 2021 (IN) 202111052413

(51) Int. Cl. B01J 31/18; B01J 37/00; C07C 5/09

(52) U.S. Cl. CPC B01J 31/1815; B01J 37/00; C07C 5/09; B01J 2231/645; B01J 2531/0244; B01J 2531/845; C07C 2531/18

(57) ABSTRACT

The present invention relates to a pincer-based cobalt catalyst of formula (I) for hydrogenation of alkynes, and its process of preparation thereof. More particularly the present invention also relates to a process for the hydrogenation of alkynes to alkenes.

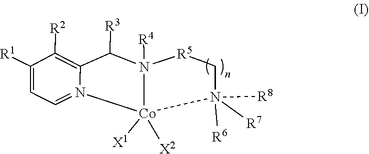

### CROSS REFERENCES TO RELATED APPLICATIONS

This application is a national-stage application under 35 U.S.C. § 371 of International Application No. PCT/IN2022/050971, filed Nov. 4, 2022, which International Application claims benefit of priority to Indian Patent Application No. 202111052413, filed Nov. 15, 2021.

### TECHNICAL FIELD

The present disclosure relates to the pincer-based cobalt catalysts for hydrogenation reaction, and its process of preparation thereof. More particularly, the present disclosure relates to pincer-based cobalt catalysts of formula (I) for hydrogenation of substituted alkynes.

### BACKGROUND

Amongst the various routes of alkene synthesis, the selective hydrogenation of alkynes is considered as the finest approach to achieve the olefins of interest. However, controlling the stereo-selectivity of resulting olefin and the over-hydrogenation to alkane are major concerns in regards to alkyne hydrogenation. Overcoming these challenges, in last few decades, various noble metal catalysts based on ruthenium, palladium and iridium are exploited for the catalytic hydrogenation of alkynes to get desired reduced olefins with specific stereochemistry (J. Am. Chem. Soc. 2002, 124, 7922; Angew. Chem. Int. Ed. 2013, 52, 355; Angew. Chem. Int. Ed. 2015, 54, 12431; J. Am. Chem. Soc. 2021, 143, 4824).

Cobalt being one of the earth-abundant transition metals with biological significance has been substantially explored for the hydrogenation of various unsaturated functional groups including nitriles. In particular, the cobalt-catalyzed semi-hydrogenation of internal alkynes to (E) or (Z)-selective alkenes using molecular hydrogen is also independently demonstrated in prior arts. In 2016, Fout reported hydrogenation of alkynes using a carbene pincer cobalt complexes and 4.0 bar hydrogen pressure (J. Am. Chem. Soc. 2016, 138, 13700). Similarly, Dong and Zhang shown hydrogenation of alkynes at 2.0 bar hydrogen (Chem. Commun. 2017, 53, 4612). Further, US20200223880A1 discloses a cobalt complex compound for the selective hydrogenation of alkenes or alkynes at the temperature range of 50-80° C. while being stirred for 10-14 hrs.

Prior literature discloses (PNP)Co(II) complexes for the semi-hydrogenation of alkynes to Z- or E-alkenes using NH₃—BH₃ as hydrogen source and even few report cobalt-catalyzed selective hydrogenation of alkynes to Z-alkenes using NH₃—BH₃ and H₂O as hydrogen source (Catal. Sci.Technol. 2018, 8, 428; Chem.Commun. 2019, 55, 5663).

Though, all these protocols efficiently provided the Z-selective alkenes from alkynes, an elevated temperature of 60-80° C. is essential and uses a phosphine-based ligand, whose synthesis is tedious involving multi-step process, and an inert atmosphere.

Therefore, there is a need in the art to develop a phosphine-free, nitrogen-ligated cobalt complex that can perform the stereoselective hydrogenation at room temperature, without requiring a special experimental set up.

The main objective of the present disclosure is to provide pincer-based cobalt catalysts of formula (I) for hydrogenation of Substituted alkynes.

Another objection of the present disclosure is to provide a process of preparation of the pincer-based catalyst of formula (I).

Another objective of the present disclosure is to provide a process for the hydrogenation of substituted alkynes by using above said pincer-based cobalt catalyst of formula (I) at 27° C.

### SUMMARY

This summary is provided to introduce a selection of concepts in a simplified form that are further described below in Detailed Description section. This summary is not intended to identify key features or essential features of the claimed subject matter, nor is it intended to be used as an aid in determining the scope of the claimed subject matter.

Accordingly, to accomplish the objectives, the present disclosure provides a pincer-based cobalt catalyst of formula (I) for hydrogenation of alkynes, specifically substituted alkynes.

In an embodiment, the present disclosure provides a pincer-based cobalt catalyst of formula (I);

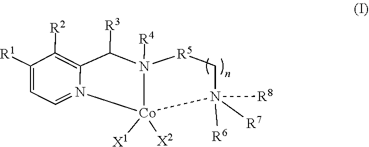

Wherein,
R¹ is selected from the group consisting of hydrogen, C₁-C₅ alkyl, alkoxy, aryl, trifluoromethyl, halogen, amino, alkylamino;
R² and R³ are selected from hydrogen or together form a cyclic arene;
R⁴ is selected from the group consisting of hydrogen, C₁-C₆ straight alkyl;
R⁵ is selected from CH₂ or C(O);
R⁶, R⁷ are each independently selected from the group consisting of C₁-C₆ straight or branched alkyl, aryl;
R⁸ is selected from hydrogen, and cyclic arene;
X¹ and X² are selected from the group consisting of Cl, Br, I, H, OTf, OC(O)CH₃;
n=0, 1, 2;
Provided, when Co—N bond is present then N—R⁸ bond is absent.

Another objection of the present disclosure is to provide a process of preparation of the pincer-based catalyst of formula (I).

Another embodiment of the present disclosure is to provide a process for the hydrogenation of substituted alkynes, alkenes and other organic compounds by using above said pincer-based cobalt catalyst of formula (I), wherein the process comprises of reacting compound of formula (5) with ammonia borane and catalyst of formula (I) in a suitable solvent at a temperature in the range of 25-30° C. for a period in the range of 15-20 hours.

### BRIEF DESCRIPTION OF THE DRAWINGS

The following drawings form part of the present specification and are included to further illustrate aspects of the present disclosure. The disclosure may be better understood by reference to the drawings in combination with the detailed description of the specific embodiments presented herein.

FIG. 1 represents thermal ellipsoid plot of catalyst 1 (Selected bond lengths (Å): Co1-N1=2.201(3); Co1-N2=02.099(3); Co1-N3=2.163(3); Co1-Cl1=2.3355(10); Co1-Cl2=2.3103(10); N1-H1=1.0. Selected bond angles (°): N1-Co1-N2=77.49(11); N3-Co1-N1=80.67(11); N1-Co1-Cl1=86.91(8); N1-Co1-Cl2=172.55(8); C1-N1-C10=112.1(3); C10-N1-Co1=107.6(2); N1-C10-C11=108.1(3).)

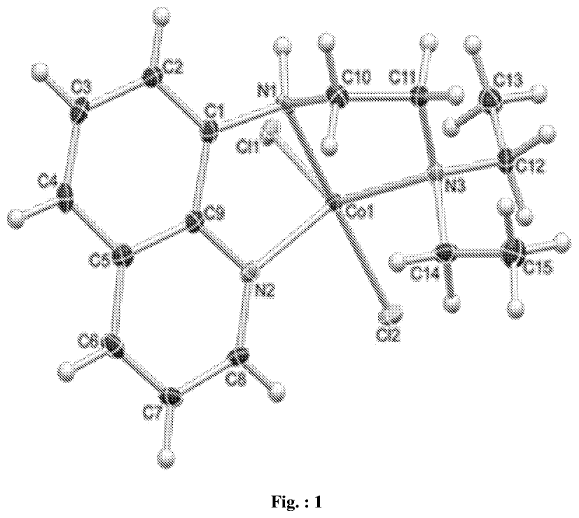

FIG. 2 represents thermal ellipsoid plot of catalyst 3. Selected bond lengths (Å): Co1-N1=1.9982(10); Co1-N2=2.0269(10); Co1-Cl1=2.2658(3); O1-C10=1.2384(14); N3-H1=0.896(18). Selected bond angles (°): N1-Co1-N2=83.09(4); N1-Co1-Cl1=113.99(3); N1-Co1-Cl2=120.46(3); C12-Co1-Cl1=105.331(12); C1-N1-C10=121.29(9); C10-N1-Co1=126.01(8); N1-C10-C11=113.68(10).

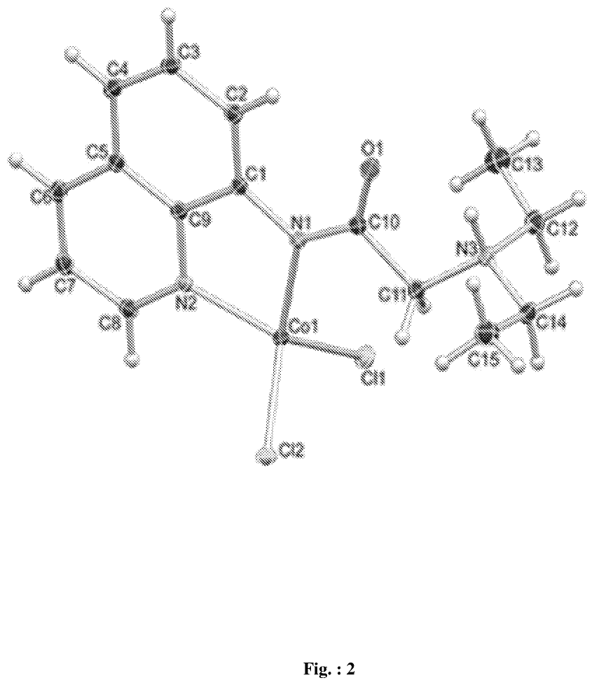

FIG. 3 is a scheme illustrating the hydrogenation reaction.

### DETAILED DESCRIPTION

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In some embodiments, numbers have been used for quantifying weights, percentages, ratios, and so forth, to describe and claim certain embodiments of the disclosure and are to be understood as being modified in some instances by the term “about.” Accordingly, in some embodiments, the numerical parameters set forth in the written description and attached claims are approximations that can vary depending upon the desired properties sought to be obtained by a particular embodiment. The numerical values presented in some embodiments of the disclosure may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context requires otherwise, throughout the specification which follow, the word “comprise” and variations thereof, such as, “comprises” and “comprising” are to be construed in an open, inclusive sense that is as “including, but not limited to.”

The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the claims. No language in the specification should be construed as indicating any non-claimed element essential to the practice of the claimed subject matter.

Groupings of alternative elements or embodiments disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified.

The description that follows, and the embodiments described therein, is provided by way of illustration of an example, or examples, of particular embodiments of the principles and aspects of the present disclosure. These examples are provided for the purposes of explanation, and not of limitation, of those principles and of the disclosure.

It should also be appreciated that the present disclosure can be implemented in numerous ways, including as a system, a method or a device. In this specification, these implementations, or any other form that the invention may take, may be referred to as processes. In general, the order of the steps of the disclosed processes may be altered within the scope of the invention.

The headings and abstract provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

In a general embodiment, the present disclosure provides a pincer-based cobalt catalyst of formula (I) for hydrogenation of substituted alkynes, alkenes and other organic compounds.

In an embodiment, the present disclosure provides a pincer-based cobalt catalyst of formula (I);

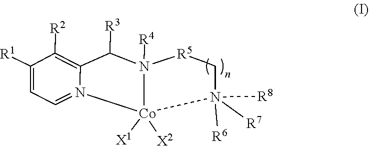

Wherein,
R¹ is selected from the group consisting of hydrogen, C₁-C₅ alkyl, alkoxy, aryl, trifluoromethyl, halogen, amino, alkylamino;
R² and R³ are selected from hydrogen or together form a cyclic arene;
R⁴ is selected from the group consisting of hydrogen, C₁-C₆ straight alkyl;
R⁵ is selected from CH₂ or C(O);
R⁶, R⁷ are each independently selected from the group consisting of C₁-C₆ straight or branched alkyl, aryl;
R⁸ is selected from hydrogen, and cyclic arene;
X¹ and X² are selected from the group consisting of Cl, Br, I, H, OTf, OC(O)CH₃;
n=0, 1, 2;
Provided, when Co—N bond is present then N—R⁸ bond is absent.

The cobalt catalyst of formula (I) is represented by:

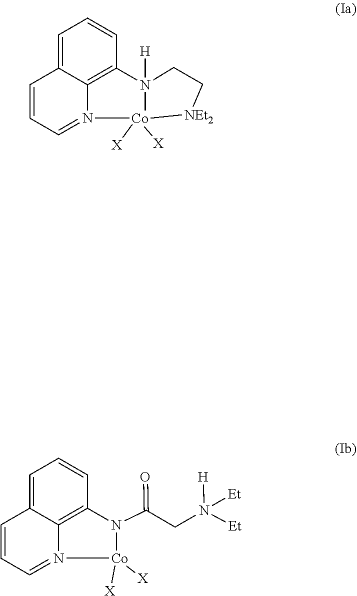

Wherein X is selected from chloro or bromo.

In another embodiment, the pincer-based cobalt catalyst of formula (I) is prepared by the process comprises of reacting anhydrous CoX₂ salt with ligand in a suitable solvent at a temperature in the range of 25-30° C. for a period in the range of 10-12 hours to afford catalyst of formula (I), wherein X is selected from group consisting of Cl, Br, I, OTf, OC(O)CH₃.

In another embodiment, the cobalt catalyst of formula (I) is prepared by the process comprises of reacting 1.0-2.0 mmol of anhydrous CoX₂ salt with 1.0-2.0 mmol of ligand in a suitable solvent at a temperature in the range of 25-30° C. for a period in the range of 10-12 hours to afford catalyst of formula (I), wherein X is selected from the group consisting of Cl, Br, I, OTf, OC(O)CH₃.

In yet another embodiment, the cobalt catalyst of formula (I) is prepared by the process comprises of reacting equal parts of anhydrous CoX₂ salt and ligand in a suitable solvent at a temperature in the range of 25-30° C. for a period in the range of 10-12 hours to afford catalyst of formula (I), wherein X is selected from the group consisting of Cl, Br, I, OTf, OC(O)CH₃.

In yet another embodiment, the cobalt catalyst of formula (I) yielded is in the range 73-95%.

In yet another embodiment, the ligand is selected from the group consisting of N1,N1-diethyl-N2-(quinolin-8-yl)ethane-1,2-diamine and 2-(diethylamino)-N-(quinolin-8-yl)acetamide.

In yet another embodiment, the solvent is selected from the group consisting of THF, toluene, dichloromethane, methanol, ethanol, and isopropanol.

Another embodiment of the present disclosure provides a process for the hydrogenation of alkynes to Z-alkenes by using above said pincer-based cobalt catalyst of formula (I), wherein the process comprises of reacting alkyne with hydrogen source and catalyst of formula (I) in a suitable solvent at a temperature in the range of 25-100° C., preferably in the range of 25-45° C. for a period in the range of 15-30 hours to afford the hydrogenated Z-alkene product.

In an exemplary embodiment, the process for the hydrogenation of substituted alkynes to Z-alkenes using the cobalt catalysts of formula (I) comprises reacting 0.5-1.0 mmol of alkyne with 0.5-1.0 mmol of a hydrogen source in presence of 0.01-0.02 mmol of the cobalt catalyst of formula (I) in a solvent at a temperature in the range of 25-100° C., preferably in the range of 25-45° C. for a period in the range of 15-30 hours to obtain Z-alkene.

In an exemplary embodiment, the process for the hydrogenation of substituted alkynes to Z-alkenes using the cobalt catalysts of formula (I) yields 68-95% with selectivity for Z-alkenes.

In another embodiment, the alkyne is selected from the group consisting of an un(substituted) diphenyl acetylene, phenyl acetylene pyridine and naphthylphenyl acetylene.

In another embodiment, the Z-alkene is selected from the group consisting of an un(substituted) diphenyl ethylene, phenyl pyridine ethene and naphthylphenyl ethene.

In another embodiment, the solvent is selected from the group consisting of ethanol, methanol, isopropanol, THF and toluene.

In another embodiment, the hydrogen source is selected from the group consisting of NH₃BH₃, Me₂NHBH₃.

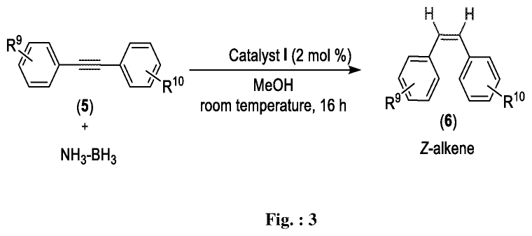

The process for the hydrogenation is depicted below in FIG. 3
wherein,
   R⁹ and R¹⁰ is selected from the group consisting of hydrogen, C₁-C₅ alkyl, alkoxy, trifluoromethyl, halogen, CH₂OH. In particularly useful embodiment, diphenyl acetylene is used as a compound of formula (5).

In yet another embodiment, the Z-alkene compounds obtained selectively by the hydrogenation process using cobalt catalyst of formula (I) are listed as below:

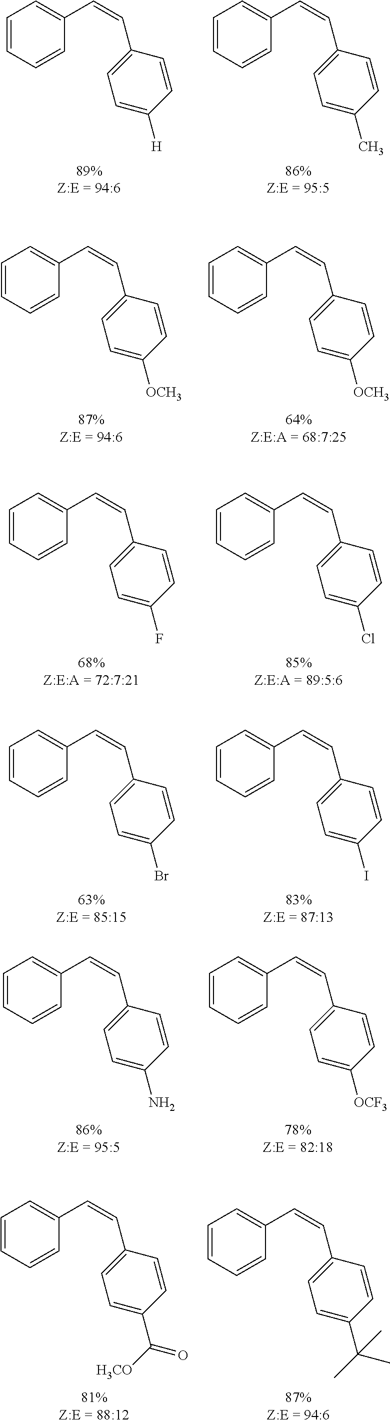

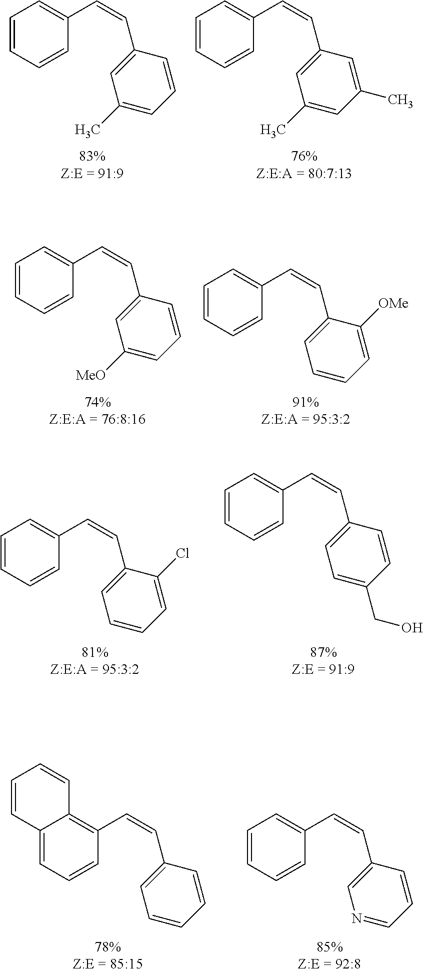

Another embodiment of the present disclosure is also to provide a process for the hydrogenation of alkenes and other organic compounds by using above said pincer-based cobalt catalyst of formula (I).

The cobalt catalyst of formula (I) performs the hydrogenation reaction at room temperature (27° C.), and this room temperature reaction makes the process advantageous, as it can tolerate sensitive functionalities like —Cl, —Br, —I, NH2, OH. Moreover, the room temperature can make the large scale process more energy efficient due to no need to heat up the reaction. This has been achieved due to structurally unique catalyst developed and discussed herein.

Further, the cobalt catalyst of formula (I) is hemilabile in nature, i.e. out of three coordination arms, one arm of the ligand is proposed to be on/off (coordinate/decoordinate) depending upon the steric and electronic requirement during the catalysis. This makes the present catalyst more active at room temperature than the ones reported in literature.

Also, the one arm can coordinate/decoordinate from cobalt depending upon the electronic requirements. That could be the reason, the present catalyst is performing the catalytic reaction at room temperature. More the substituents and ligand backbone is totally different in the present catalyst, which makes it a hemilabile catalyst.

The term, “alkyl” or “C₁-C₅ alkyl” or “C₁-C₆ alkyl” or “alkyl in alkylamino” as used herein, refers to the radical of saturated aliphatic groups, including straight or branched-chain alkyl groups having six or fewer carbon atoms in its backbone, for instance, C₁-C₅ or C₁-C₆ alkyl for straight chain and C₃-C₆ for branched chain. As used herein, C1-C5 alkyl refers to an alkyl group having from 1 to 5 carbon atoms and C₁-C₆ alkyl refers to an alkyl group having from 1 to 6 carbon atoms. Representative examples of alkyl include, but are not limited to, methyl, ethyl, n-propyl, n-butyl, n-pentyl, n-hexyl, isopropyl, sec-butyl, isobutyl, tert-butyl, 2-methylbutyl, 3-methylbutyl and so on.

Furthermore, unless stated otherwise, the alkyl group can be unsubstituted or substituted with one or more substituents, for example, from one to four substituents, independently selected from the group consisting of halogen, hydroxy, cyano, nitro and amino. Examples of substituted alkyl include, but are not limited to hydroxymethyl, 2-chlorobutyl, trifluoromethyl and aminoethyl.

The term, “halogen” as used herein refers to chlorine, fluorine, bromine or iodine atom.

The term, “alkoxy” refers to a (C₁-C₆) alkyl having an oxygen radical attached thereto. Representative examples of alkoxy groups include, but are not limited to, methoxy, ethoxy, propoxy, isopropoxy, n-butoxy, isobutoxy, tert-butoxy, n-pentoxy, and so on. Furthermore, unless stated otherwise, the alkoxy groups can be unsubstituted or substituted with one or more groups. A substituted alkoxy refers to a (C₁₋₆)alkoxy substituted with one or more groups, particularly one to four groups independently selected from the groups indicated above as the substituents for the alkyl group.

The term “C₁-C₁₀ cycloalkyl” refers to a saturated or unsaturated cyclic hydrocarbon radical including 1, 2 or 3 rings and including a total of 3-10 carbon atoms forming the rings. Examples include, but are not limited to, cyclopropyl, cyclobutyl, cyclopentyl, cyclohexyl, adamantyl, norbornyl, bicyclo[2.1.0]pentane, cyclopentenyl, cyclohexenyl, cycloheptyl, cyclooctyl, cyclononyl and so on. Unless stated otherwise, said cycloalkyls can be unsubstituted or substituted with one or more substituents, for example, substituents independently selected from the group consisting of oxo, halogen, (C₁₋₁₀)alkyl, hydroxy, cyano, nitro and amine. Cycloalkyl group comprises a saturated cycloalkyl ring system which does not contain any double bond within the ring or a partially unsaturated cycloalkyl ring system which may contain one or more double bonds within the ring system that is stable, and do not form an aromatic ring system.

The term “aryl” or “arene” or “cyclic arene” as used herein refers to monocyclic, bicyclic or tricyclic hydrocarbon groups having 6 to 14 ring carbon atoms, wherein at least one carbocyclic ring is having a g electron system. Examples of aryl ring systems include, but are not limited to, phenyl, benzyl, naphthyl, anthracenyl, phenanthrenyl and so on. Unless indicated otherwise, aryl group can be unsubstituted or substituted with one or more substituents, for example 1-4 substituents independently selected from the group consisting of halogen, (C₁-C₁₀)alkyl, (C₁-C₆)alkoxy, acetyl, 9H-carbazol-9-yl, hydroxy, phenyl, cyano, nitro, —COOH and amino. The substituents can be present on either the ring carbon or the ring nitrogen atom(s). The substituents can be present at one or more positions provided that a stable molecule results.

The term “(un)substituted” in (un)substituted diphenylacetylene as used herein refers to either no substitutions or substitutions on one or both phenyl rings of diphenylacetylene, where the substitutions include, but are not limited to, alkyl, halogen, CF3, amino, OCF3, ester, hydroxyl, alkoxy, cycloalkyl, aryl, heteroaryl, and so on, as detailed above.

### EXAMPLES

Following examples are given by way of illustration and therefore should not be construed to limit the scope of the claimed subject matter.

### Example 1: General Process for the Synthesis of Cobalt Catalyst of Formula (I)

A solution of ligand (0.243 g, 1.0 mmol) in THF (10 mL) was added dropwise to the anhydrous CoCl₂ (0.130 g, 1.0 mmol) in THF (10 mL) and the reaction mixture was stirred at 27° C. for 12 h. The solid precipitate obtained was filtered and washed with Et₂O (5 mL×3). Upon drying under vacuum, the catalyst was obtained.

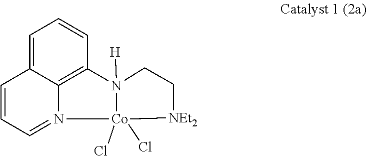

Yield: 0.339 g, 91%. The crystal suitable for a single-crystal X-ray diffraction was obtained from saturated solution of catalyst 2a in acetonitrile at −15° C. Elemental Analysis Calcd (%) for C, 48.28; H, 5.67; N, 11.26; Found: C, 48.36; H, 5.24; N, 10.75.

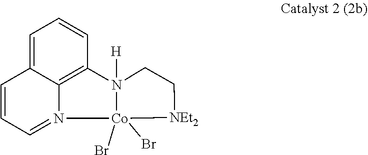

Yield: 0.430 g, 93%. Elemental Analysis Calcd (%) for C, 38.99; H, 4.58; N, 9.09; Found: C, 38.56; H, 4.78; N, 9.47.

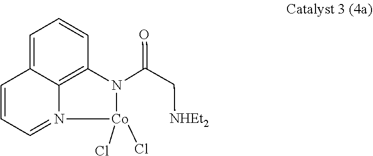

Yield: 0.364 g, 94%. The crystal suitable for a single-crystal X-ray diffraction was obtained from saturated solution of catalyst 4a in acetonitrile at −15° C. Elemental Analysis Calcd (%) for C, 46.53; H, 4.95; N, 10.85. Found: C, 46.04; H, 4.64; N, 10.63.

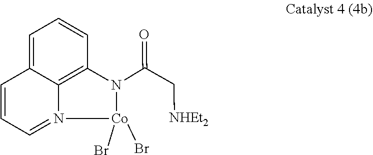

Yield: 0.452 g, 95%. Elemental Analysis Calcd (%) for C, 37.84; H, 4.02; N, 8.83. Found: C, 37.52; H, 3.75; N, 9.09.

### Example 2: Representative Procedure for Hydrogenation

A Teflon screw-cap tube was introduced with catalyst I (0.0048 g, 0.01 mmol), ammonia borane (0.0155 g, 0.5 mmol) and diphenylacetylene (0.089 g, 0.5 mmol) inside the glove box. The solvent MeOH (1.5 mL) was added to the reaction vessel under the argon atmosphere. The reaction mixture was then stirred at 27° C. for 16 h. At ambient temperature, the reaction mixture was diluted with MeOH (5.0 mL) and resulting solution was concentrated under vacuum. The crude reaction mixture was then purified by flash chromatography using petroleum ether as eluent to obtain Z-stilbene (0.080 g, 89%).

Characterization Data for (Z)-1,2-diphenylethene: The representative procedure was followed, using 1,2-diphenylethyne (0.089 g, 0.50 mmol), NH₃—BH₃ (0.0155 g, 0.5 mmol) and catalyst I (0.0048 g, 0.01 mmol). Purification by column chromatography on silica gel (petroleum ether) yielded (Z)-1,2-diphenylethene (0.080 g, 89%) as a colourless oil. ¹H-NMR (500 MHz, CDCl₃): δ 7.29-7.18 (m, 10H, Ar—H), 6.63 (d, J=7.6 Hz, 2H, CH). ¹³C{¹H}-NMR (125 MHz, CDCl₃): δ 137.4 (2C, C_{q}), 130.4 (2C, CH), 129.1 (4C, CH), 128.4 (4C, CH), 127.3 (2C, CH). HRMS (ESI): m/z Calcd for C₁₄H₁₂+H⁺ [M+H]⁺ 181.1017; Found 181.1012.

In this disclosure, a phosphine-free catalyst has been developed for the hydrogenation process.

In this disclosure, a hydrogenation process takes place at 25-30° C.

This disclosure provides an indigenously developed catalyst.

From the foregoing, it will be appreciated that, although specific embodiments of the invention have been described herein merely for purposes of illustration, it will be evident to those skilled in the art that numerous variations of the details may be made without departing from the intended scope of the appended claims.

1-11. (canceled)

12. A pincer-based cobalt catalyst for hydrogenation of alkynes to alkenes, the pincer-based cobalt catalyst having formula (I): 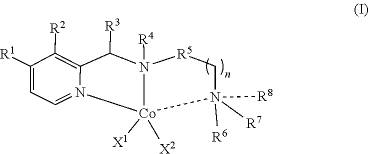
where:
R¹ is selected from the group consisting of hydrogen, C₁-C₅ alkyl, alkoxy, aryl, trifluoromethyl, halogen, amino, and alkylamino;
R² and R³ are either both hydrogen or together form a cyclic arene;
R⁴ is selected from the group consisting of hydrogen and C₁-C₆ alkyl;
R⁵ is selected from —CH₂ or —C(O);
R⁶ and R⁷ are each independently selected from the group consisting of C₁-C₆ alkyl and aryl;
R⁸ is selected from hydrogen and cyclic arene;
X¹ and X² are selected from the group consisting of —Cl, —Br, —I, —OTf, and —OC(O)CH₃;
n is 0, 1, or 2; and
when a Co—N bond is present, a N—R⁸ bond is absent.

13. The pincer-based cobalt catalyst of claim 12, wherein catalyst of formula (I) is represented by formula (Ia) or formula (Ib): 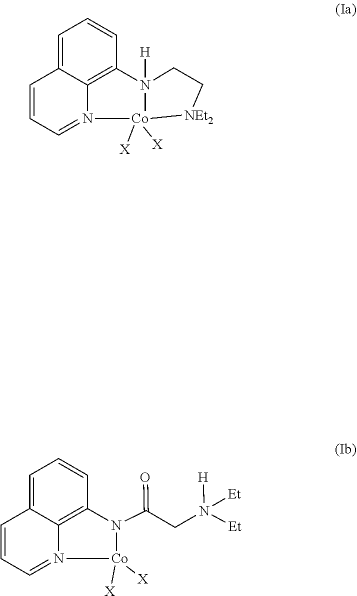
wherein each X in formula (Ta) and formula (Tb) is chloro or bromo.

14. A process for preparing the pincer-based cobalt catalyst according to claim 12, the process comprising:
(i) reacting an anhydrous CoX₂ salt with a ligand in a solvent to obtain a mixture, wherein X is selected from the group consisting of Cl, Br, I, OTf, OC(O)CH₃; and
(ii) heating the mixture at a temperature from 25° C. to 30° C. for 10 hours to 12 hours to obtain the pincer-based cobalt catalyst.

15. The process of claim 14, wherein the ligand is selected from the group consisting of N¹,N¹-diethyl-N²-(quinolin-8-yl)ethane-1,2-diamine and 2-(diethylamino)-N-(quinolin-8-yl)acetamide.

16. The process of claim 14, wherein the solvent is selected from the group consisting of tetrahydrofuran, toluene/dichloromethane, methanol, ethanol, and isopropanol.

17. A process for the hydrogenation of alkynes to Z-alkenes using the pincer-based cobalt catalyst according to claim 12, the process comprising:
(i) reacting an alkyne and a hydrogen source in presence of the pincer-based cobalt catalyst to obtain a reaction mixture; and
(ii) heating the reaction mixture of (i) in a solvent at a temperature from 25° C. to 100° C. for 15 hours to 30 hours to obtain a Z-alkene.

18. The process of claim 17, wherein the temperature is from 25° C. to 45° C.

19. The process of claim 17, wherein:
the alkyne is selected from the group consisting of (un)substituted diphenyl acetylene, phenyl acetylene pyridine, and naphthylphenyl acetylene; and
the Z-alkene is selected from the group consisting of (un)substituted diphenyl ethylene, phenyl pyridine ethane, and naphthylphenyl ethene.

20. The process of claim 19, wherein the Z-alkenes are selected from:

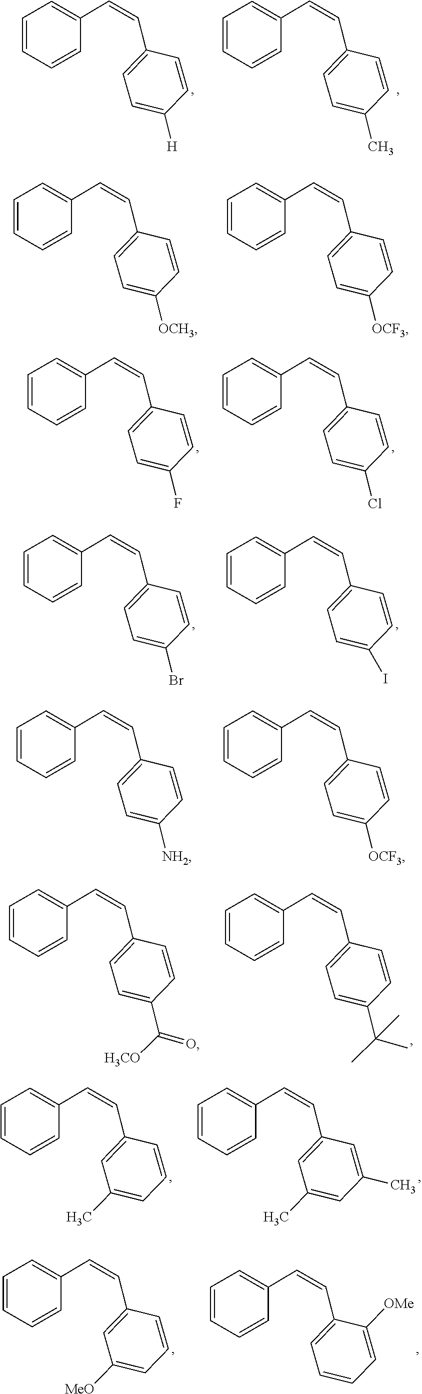

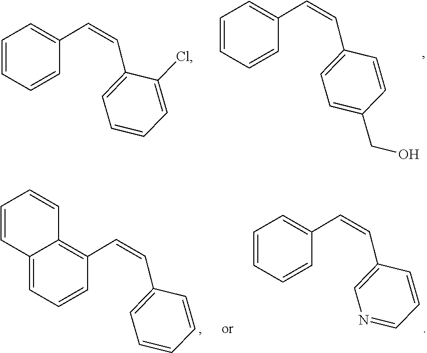

21. The process of claim 17, wherein the solvent is selected from the group consisting of ethanol, methanol, isopropanol, tetrahydrofuran, and toluene.

22. The process of claim 17, wherein the hydrogen source is selected from the group consisting of NH₃BH₃ and Me₂NHBH₃.
